(19) United States
(12) Patent Application Publication
(10) Pub. No.: US 2026/0346007 A1
(43) Pub. Date: Nov. 5, 2026

(54) LOW-VOLTAGE ELECTROSTIMULATION DEVICES AND METHODS FOR VENOM PROTEIN DENATURATION AT BITE SITES

(71) Applicant: Seldon Advisory LLC, Alpine, NJ (US)

(72) Inventors: Sandra Nudelman, Alpine, NJ (US); Avi Nudelman, Boynton Beach, FL (US)

(21) Appl. No.: 19/197,897

(22) Filed: May 2, 2025

(51) Int. Cl. A61N 1/36; A61N 1/04; A61N 5/06

(52) U.S. Cl. CPC A61N 1/36034; A61N 1/0476; A61N 1/36031; A61N 5/0625; A61N 2005/0659; A61N 2005/0661

(57) ABSTRACT

A portable device for denaturing venom proteins via controlled low-voltage electrostimulation at a bite site. The device includes a housing with a central electrode and one or more surrounding electrodes, a voltage control circuit generating an output voltage, and an electronic switch for delivering current through the electrodes. Optional features include pulse modulation, polarity reversal, ultraviolet light emission, infrared heat, and impedance-based bite site detection. The portable device may be positioned over a bite site, such that low-voltage pulses are applied to the bite site to disrupt venom proteins while optionally exposing the site to germicidal ultra-violent light and infrared heat to reduce inflammation.

### FIELD OF INVENTION

The embodiments relate to devices for treating venomous bites, and more particularly to a venom treatment device for denaturing venom proteins using low-voltage electrical current.

### BACKGROUND

Venomous bites from snakes, insects, and other animals pose a significant health risk worldwide. The bites can cause severe pain, tissue damage, and, in some cases, life-threatening complications to various species, including humans, other animals, and birds. Traditional treatments for venomous bites often involve antivenom administration, which may have limitations such as immediate availability, cost, and potential adverse reactions.

Electrical stimulation has been explored as an alternative approach for treating venomous bites. Some studies suggest that applying electrical current to the bite site may help denature venom proteins and reduce their toxic effects. However, existing electrical devices for this purpose typically use high-voltage shocks or inconsistent voltage, which can be painful and potentially harmful to tissue.

Heat treatment has also been explored as a potential method for addressing venomous bites. However, the application of heat alone to the affected area may cause discomfort or pain, especially if the bite site is already inflamed or sensitive. Additionally, heat treatment may require precise temperature control to be effective without causing tissue damage, which can be challenging to achieve in field conditions. In some cases, excessive heat application may accelerate the spread of venom through increased blood flow to the area. Furthermore, the effectiveness of heat treatment may vary depending on the type of venom and the specific proteins involved, potentially limiting its universal applicability across different types of venomous bites.

Piezoelectric devices have also been investigated for venom treatment applications. These devices may generate an electrical charge in response to mechanical stress, potentially providing a portable and battery-free option for electrical stimulation. However, piezoelectric devices may have limitations in the context of venom treatment. The voltage output from piezoelectric elements may be inconsistent and difficult to control precisely, potentially leading to unpredictable treatment outcomes. Additionally, the mechanical action required to generate the electrical charge may cause discomfort or further tissue damage when applied to an already sensitive bite area. In some cases, the electrical output from piezoelectric devices may be insufficient to effectively denature venom proteins, particularly for deeper tissue envenomation. Furthermore, the durability and reliability of piezoelectric elements in field conditions may be a concern, as exposure to moisture or extreme temperatures could affect their performance.

Portable, user-friendly devices for administering electrical treatment at the bite site are therefore desirable, especially in remote areas where medical care may not be readily accessible. Such devices could potentially provide rapid intervention to mitigate venom effects before more comprehensive medical treatment can be obtained.

### BRIEF DESCRIPTION OF FIGURES

FIGS. 1A-C illustrate an electrostimulation device, according to some embodiments.

FIG. 2 illustrates a circuit diagram for an electrostimulation device, according to some embodiments.

FIGS. 3A-C illustrate a section view of an electrostimulation device, according to some embodiments.

FIG. 4 illustrates another circuit diagram of an electrostimulation device, according to some embodiments.

FIGS. 5A-C illustrate a perspective view of an electrostimulation device, according to some embodiments.

FIG. 6 illustrates a circuit diagram for an electrostimulation device, according to some embodiments.

FIG. 7 illustrates a flowchart of a method for treating venom using an electrostimulation device, according to some embodiments.

Embodiments of the disclosure and their advantages are best understood by referring to the detailed description that follows. It should be appreciated that like reference numerals are used to identify like elements illustrated in one or more of the figures, wherein showings therein are for purposes of illustrating embodiments of the disclosure and not for purposes of limiting the same.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

The embodiments are directed to electrostimulation devices, such as a venom treatment device used for treating venom from insect (e.g., mosquito, bees, etc.) or snake bites at a bite site in humans or other warm-blooded species. The venom treatment device applies a continuous low voltage to an affected area using one or more electrodes, causing the venomous proteins to break down, potentially reducing toxins on the surface and underneath the skin at the bite site. The voltage may be approximately between 9 and 15 volts, though the implementation may not be limited to these embodiments, and may be within other ranges sufficient to alter protein folding without causing dermal burns. The use of a low-voltage venom treatment device for treating venomous bites may offer several advantages over conventional devices that use high-voltage, heat dissipation, and piezoelectrics. The low-voltage venom treatment device may be more effective at denaturing venom proteins without causing additional tissue damage or pain to the affected area. The controlled application of low-voltage current may disrupt the molecular structure of venom proteins, potentially reducing their toxic effects on the body. Additionally, the low-voltage venom treatment device are portable and may be carried to remote areas to provide timely treatment to insect or snake bites.

Furthermore, low-voltage devices may provide a safer treatment option, especially in field conditions where medical supervision may not be immediately available. In some cases, the lower voltage output generated by the venom treatment device may reduce the risk of electrical burns or other complications associated with high-voltage shock treatments. Additionally, the use of low-voltage stimulation may allow for longer treatment durations, which may enhance the overall effectiveness of the venom protein denaturation process without compromising patient comfort or safety. The venom protein denaturation process may be an irreversible structural change of venom proteins, resulting in loss of toxic activity, as induced by electrical stimulation, such as stimulation caused by low-voltage devices described herein.

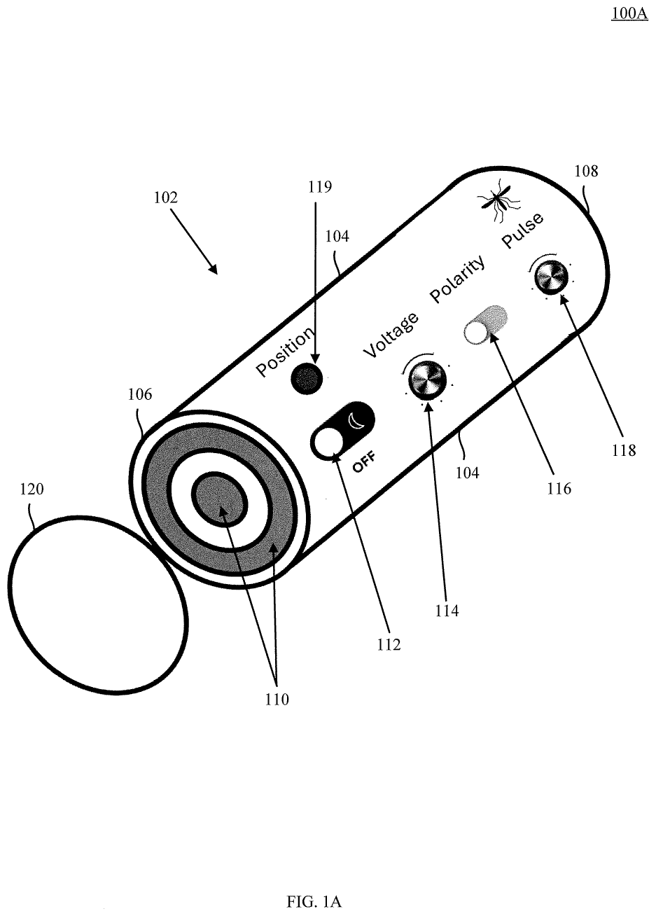

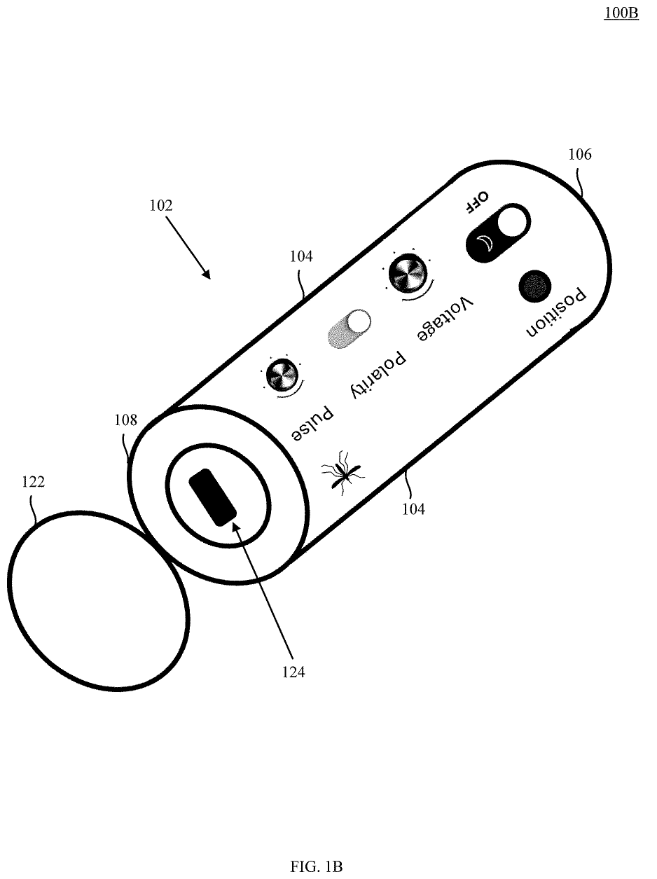

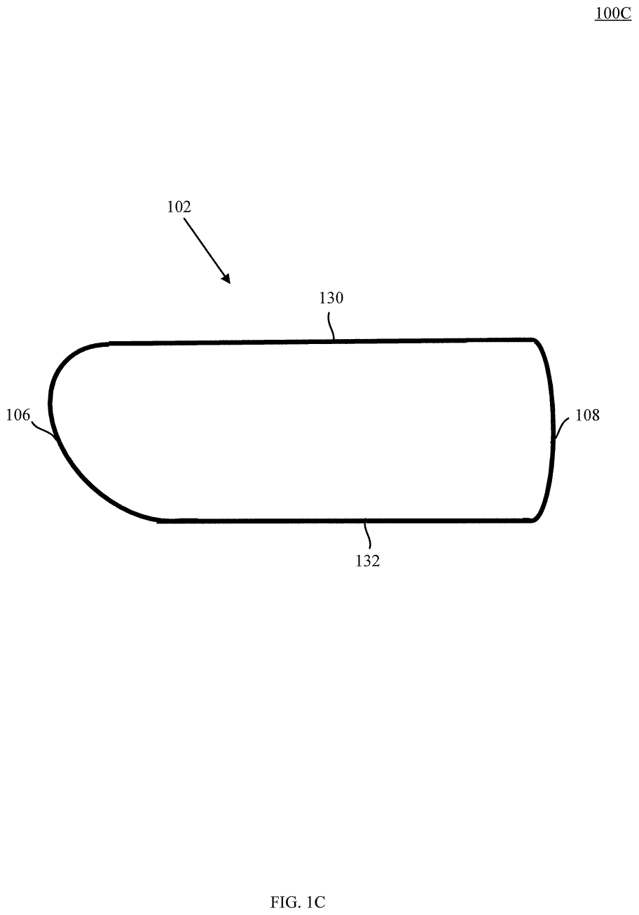

FIGS. 1A-C are diagrams 100A-C of an example electrostimulation device, according to some embodiments. An electrostimulation device 102 shown in FIGS. 1A-C may be a venom treatment device for treating insect bites, snake bites, and the like, at a bite site. Electrostimulation device 102 may have a generally elongated shape with rounded edges, providing a comfortable and ergonomic form factor for handheld use. In some aspects, electrostimulation device 102 may include a device housing 104 that may taper slightly from one end to the other, creating a sleek profile that facilitates easy gripping and manipulation. The front of the device 106 may feature a slightly convex surface to accommodate the arrangement of electrodes, while the back of the device 108 may have a more flattened profile for stability when placed on a surface. The overall shape may be designed to balance portability with functional requirements, allowing for easy storage and transport while providing sufficient space for internal components and user interface elements.

The front of the device 106 may incorporate one or more electrodes 110 arranged in a configuration to facilitate effective voltage delivery to a bite site. In some cases, the electrodes 110 may be in a configuration with a circular center followed by concentric rings, where the circular center is configured to be placed in a center of the bite area. In other cases, the electrodes 110 may include parallel bars (not shown) that are placed on a bite area. Electrodes 110 may also be configured in various patterns, such as a central delivery pin and surrounding delivery pins or a contiguous metal ring, a linear array, or other geometric layouts to optimize contact with the skin surface. The electrodes 110 configurations may also vary depending on the bite type. For example, electrodes 110 may be adjustable or interchangeable, enabling customization based on the specific treatment requirements or bite characteristics. The integration of the electrodes 110 on the front of the device 106 may allow for direct and controlled application of the low-voltage electrical stimulation to the targeted area, e.g., a bite site, potentially enhancing the efficacy of venom protein denaturation.

Electrostimulation device 102 may also include various controls, including a power control 112, a voltage control 114, a polarity control 116, and a pulse control 118. These various controls 112-118 may be positioned on one of the elongated surfaces of the device housing 104.

The power control 112 may be a button, switch, or other user-operable interface element that allows users to turn the electrostimulation device 102 on and off. In certain implementations, the power control 112 may incorporate an LED indicator to visually display the device's power status. The positioning of the power control 112 on the device housing 104 may be optimized for ergonomic use, allowing users to activate or deactivate the device quickly in emergency situations.

The voltage control 114 may allow users to adjust the low-voltage output of the electrostimulation device 102. In some aspects, the voltage control 114 may be implemented as a rotary dial, slider, or digital interface that enables precise selection of voltage levels within a predetermined range suitable for venom protein denaturation. The voltage control 114 may offer incremental adjustments, allowing users to fine-tune the electrical output based on factors such as bite severity, venom type, or patient sensitivity.

In some cases, the voltage control 114 may include preset voltage settings corresponding to different types of venomous bites or treatment protocols. These presets may be based on empirical data or clinical guidelines for treating specific types of envenomation. The voltage control 114 may also incorporate safety features to prevent accidental selection of voltages outside the intended therapeutic range, such as between 9 and 15 volts. In some embodiments (not shown), the interface of the voltage control 114 may include clear markings or a digital display to indicate the selected voltage level, enhancing user comprehension and facilitating proper device operation.

The polarity control 116 may allow users to adjust the direction of electrical current flow through the electrodes 110. In some aspects, the polarity control 116 may be implemented as a switch or button that enables users to reverse the polarity of the electrical stimulation. In other aspects the polarity control 116 may reverse polarity automatically at various treatment intervals, such as every 5 or 15 seconds, or another configurable number of seconds. This functionality may be useful in certain treatment scenarios where alternating the direction of current flow could enhance the effectiveness of venom protein denaturation.

The polarity control 116 may be designed with clear visual indicators to show the current polarity setting, helping users to adjust the treatment parameters easily. In some implementations, the polarity control 116 may be integrated with the device's microcontroller or circuit assembly to automatically alternate polarity at predetermined intervals, potentially optimizing the treatment process without requiring user intervention.

The ability to control polarity may offer additional flexibility in addressing different types of venomous bites, as some venom proteins may respond more effectively to specific current directions. The polarity control 116 may also work in conjunction with other device controls, such as the voltage control 114 and pulse control 118, to provide a comprehensive range of adjustable parameters for tailoring the electrical stimulation to treatment needs of a specie.

The pulse control 118 may allow users to adjust the timing and pattern of electrical stimulation delivered through the electrodes 110. In some aspects, the pulse control 118 may be implemented as a dial, button, or digital interface that enables users to modify pulse parameters such as frequency, duration, and duty cycle. The pulse control 118 may offer various preset pulse patterns optimized for different types of venomous bites or treatment protocols. Users may be able to select from continuous voltage stimulation, intermittent pulses, or more complex waveforms. In some implementations, the pulse control 118 may allow for fine-tuning of individual pulse characteristics, such as rise time, fall time, and inter-pulse interval. During the portions of the pulse, the constant low-voltage stimulation may be delivered to the bite site via electrodes 110.

The ability to adjust pulse parameters may provide several potential benefits in venom treatment. Pulsed stimulation may help prevent tissue fatigue and maintain treatment efficacy over longer periods. Different pulse patterns may also target specific venom proteins more effectively or enhance the overall denaturation process.

In some cases, the pulse control 118 may work in conjunction with other device controls to create comprehensive treatment profiles. For example, users may be able to combine specific voltage levels, polarity settings, and pulse patterns to address particular types of envenomation. The pulse control 118 may include visual indicators or a digital display to clearly communicate the current pulse settings to the user.

The electrostimulation device 102 may include a bite site identifier 119, which may be implemented as an LED light or other visual indicator. In some aspects, the bite site identifier 119 may work in conjunction with a current sensor integrated into the electrostimulation device 102. This current sensor may be configured to detect variations in electrical impedance across the skin surface, potentially identifying areas of elevated current impedance that may correspond to the bite site or areas of venom concentration also known referred to as bite site impedance. Bite site impedance may be characterized by elevated electrical impedance (as compared to other areas on the skin without the bite) or above a configurable impedance threshold and is detectable at or near the site of envenomation, due to inflammation or tissue disruption. The configurable impedance threshold may be programmed or pre-programmed into device 102 and maybe stored in memory of device 102. In some instances, a table of programmed or pre-programmed configurable impedance thresholds mapping to different skin types or venom classifications may be stored in device 102, from which one of impedance thresholds maybe be selected. When an optimal treatment area is identified based on these impedance measurements, the bite site identifier 119 may illuminate or change color to alert the user. In some implementations, the device 102 may also incorporate an audio alert feature that works in tandem with or instead of the visual indicator. This audio alert may emit a tone or series of beeps to guide the user towards the optimal treatment area. The combination of visual and auditory feedback may enhance the user's ability to accurately position the electrodes 110 for maximum treatment efficacy, potentially improving the overall effectiveness of the venom protein denaturation process. In some cases, bite site identifier 119 may be an ultra-violet (UV) light source with a UV emitting diode or bulb (e.g., UV-A, UV-B, or UV-C) that may be directed at a bite site to inactivate bacteria, viruses and/or other pathogens. In some instances, the bacteria, viruses, and/or other pathogens may be inactivated in less than or approximately ten seconds. In addition to disinfecting the bite site, bite site identifier 119 may also prevent or reduce the likelihood of bacteria, virus, and/or pathogen transmission when the electrostimulation device 102 is applied to multiple individuals and may be activated during or after stimulation cycle when electrodes 110 are applied to the bite site.

The front cover 120 may serve as a protective element for the electrodes 110 when the electrostimulation device is not in use. In some aspects, the front cover 120 may be designed to snap or slide into place over the electrodes 110, shielding them from dust, moisture, or physical damage during transport or storage. The front cover 120 may be constructed from a durable, non-conductive material that complements the overall design of the device housing 104. In certain implementations, the front cover 120 may incorporate a locking mechanism to ensure secure attachment to the device. When removed, the front cover 120 may be designed to attach to the back of the device or be stored separately, allowing for unobstructed access to the electrodes 110 during treatment. The integration of the front cover 120 may enhance the longevity and reliability of the electrodes 110, potentially contributing to the consistent performance of the electrostimulation device over time.

FIG. 1B shows a side and bottom view of the electrostimulation device, according to some embodiments. As illustrated in FIG. 1B, the back of device 108 may include a charge port 124. The charge port 124 may be used to connect the electrostimulation device 102 to a power source or charge an internal power source, such as, but not limited to, a lithium battery. In some embodiments, the charge port 124 may be a USB charge port. This configuration may allow for convenient charging of the electrostimulation device 102 using standard USB cables and power adapters. The USB charge port may support various USB standards, such as USB-C or micro-USB, providing compatibility with a wide range of charging accessories. Additionally, the use of a USB charge port may enable data transfer capabilities, potentially allowing for device firmware updates or treatment data logging, updating control parameters, e.g., voltage ranges, pulse duration, etc., when connected to a computer or other compatible devices.

In some embodiments, the charge port 124 may be designed to accommodate battery replacement, such as, but not limited to, for a lithium battery. The charge port 124 may include a removable cover or panel that, when opened, provides access to a battery compartment within the device housing 104. This battery compartment may be configured to securely hold the battery or other suitable power source.

In some aspects, the charge port 124 may include a dual-function design that allows for both external charging and battery replacement. This configuration may provide users with flexibility in powering the electrostimulation device, allowing them to quickly replace batteries in field situations where charging may not be feasible. The charge port 124 may also include weather-resistant seals or gaskets to protect the internal components from moisture or debris when the battery compartment is accessed.

The electrostimulation device 102 may include circuitry to automatically detect the power source type (e.g., rechargeable battery vs. replaceable 9V battery) and adjust its charging and power management functions accordingly. This may allow users to seamlessly switch between rechargeable and replaceable battery options based on their specific needs or circumstances.

Back cover 122 may be designed to cover and protect the charge port 124 when not in use. The cover may be constructed from a durable, flexible material that allows it to securely snap or slide into place over the charge port 124, shielding it from dust, moisture, and physical damage. In certain implementations, the back cover 122 may be tethered to the device housing to prevent loss while still allowing easy access to the charge port 124 when needed. The cover may also incorporate a weather-resistant seal to enhance the overall durability of the device, potentially extending its usability in various environmental conditions. When opened, the back cover 122 may be designed to fold or rotate out of the way, providing unobstructed access to the charge port 124 for convenient charging or power connection.

FIG. 1C depicts a side view of the electrostimulation device, according to some embodiments. The electrostimulation device 102 may have an upper surface 130 and a lower surface 132, which may define the overall shape of the device.

In some aspects, the controls of FIG. 1A, including the power control 112, voltage control 114, polarity control 116, and/or pulse control 118, may be arranged along the flat upper surface 130 of the electrostimulation device 102. This configuration may allow for easy access and manipulation of the controls 112-118 while the electrostimulation device 102 is held in various orientations. The placement of controls on the upper surface 130 may enhance user ergonomics and facilitate one-handed operation of the device 102.

The front of the device 106 may be angled relative to the lower surface 132. In some implementations, this angle may be approximately 45 degrees, although other angles may be used depending on the specific design requirements and ergonomic considerations. The angled configuration of the front of the device 106 may facilitate easier application of the electrodes 110 to the affected area. This design may allow users to maintain a comfortable grip on the device while positioning the electrodes 110 against the skin surface at an optimal angle for treatment delivery. The angled front may also improve visibility of the treatment area during application, potentially enhancing the accuracy and effectiveness of the venom treatment process.

The materials used in the electrostimulation device 102 are biocompatible and suitable for dermal contact.

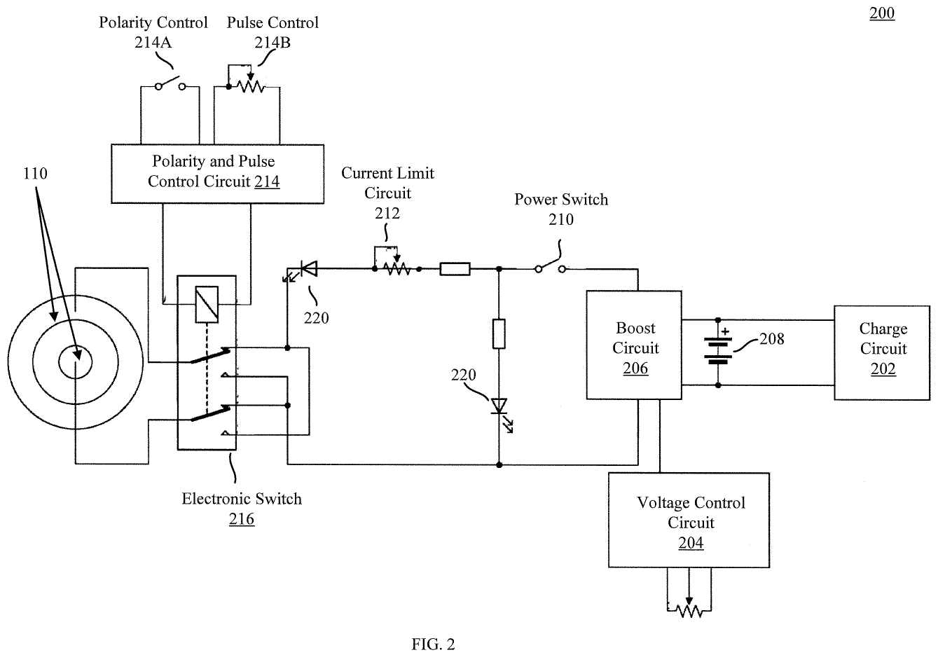

FIG. 2 illustrates an example circuit assembly 200 for a electrostimulation device, according to some embodiments. The circuit assembly 200 may include various interconnected components for controlling electrical stimulation delivery. In some embodiments, the circuit assembly 200 may include a charge circuit 202, a variable voltage control 204, a boost circuit 206, and a battery assembly 208.

The charge circuit 202 may be connected to the battery assembly 208 to manage power storage and delivery to other components in the circuit assembly 200. In some cases, the charge circuit 202 may interface with the charge port 124 of FIG. 1C to allow for recharging of the battery assembly 208.

The voltage control circuit 204 may be connected between the battery assembly 208 and the boost circuit 206. The voltage control circuit 204 may allow for adjustment of the output voltage based on inputs provided via voltage control 114 discussed in FIG. 1A. In some implementations, the voltage control circuit 204 may be configured to generate a low-voltage output between or in the range of 9V and 15V which may be sufficient to alter protein folding without causing dermal burns on the surface (e.g., skin). Notable other voltage outputs may also be generated depending on a type of protein and/or venom at the bite site. In yet further embodiments, the low-voltage output may be a continuous voltage output.

The boost circuit 206 may be connected to the battery assembly 208 and may enable variable voltage control functionality. In some cases, the boost circuit 206 may modify the voltage from the battery assembly 208 to achieve desired output voltage levels. In some instances, boost circuit 206 may include an inductor that may store voltage from the battery that is then released to voltage control circuit 204.

A power switch 210 may control the overall power delivery to the circuit. The power switch 210 may be operatively connected to the power control 112 on the device housing 104 discussed in FIG. 1A. When power switch 210 is activated, the circuit assembly 200 may deliver the low-voltage output through the electrodes 110 to denature at least one venom protein.

A current limit circuit 212 may be positioned to regulate the current flow through the circuit assembly 200 to provide continuous and/or low-voltage stimulation via electrodes 110. For example, current limit circuit 212 may regulate current flow to generate fixed current that may be set by a user to generate a voltage set by voltage control 114.

A polarity and pulse control circuit 214 may be connected to the current limit circuit 212. Polarity and pulse control circuit 214 may include polarity control 214A and pulse control 214B. The polarity and pulse control circuit 214 may manage the electrical signal delivery to the electrodes. In some implementations, the polarity and pulse control circuit 214 may be configured to reverse polarity of the low-voltage output based on polarity control 116 via polarity control 214A and generate pulses of a duration set using pulse control 118 discussed in FIG. 1A via pulse control 214B.

An electronic switch 216 may be connected to the polarity and pulse control circuit 214. The electronic switch 216 may control the routing of current between the electrodes 110 based on signals from the polarity and pulse control circuit 214. In some cases, the electronic switch 216 may be configured to control current delivery to the electrodes 110 that would correspond to a low voltage specified by voltage control circuit 204. In some embodiments, when low-voltage stimulation is delivered by electrodes 110, all electrodes 110 may be activated for a continuous time period, such as 10 or 15 seconds, or in the range of 10 to 30 seconds. In other embodiments, when the low-voltage stimulation is delivered in pulses, all electrodes 110 may be activated for the duration of a pulse or a portion of the pulse, or some electrodes may deliver low-voltage for a first portion of the pulse, while other electrodes may deliver a low voltage for a second portion of the pulse.

The circuit assembly 200 may include one or more light indicators 220 comprising an LED, UV emitting diodes, etc., and associated components to provide visual feedback about the device status (e.g., device on, off, ready to disperse voltage), or to the bite site identifier 119 for optimal placement of the electrostimulation device 102 on a bite site.

The components of the circuit assembly 200 may be arranged to allow for controlled delivery of electrical stimulation, with monitoring and adjustment of current and voltage parameters. A person of skill in the art may further appreciate that the above circuit assembly 200 is exemplary and that other types of circuit assemblies may also be designed to provide a low voltage to electrodes 110.

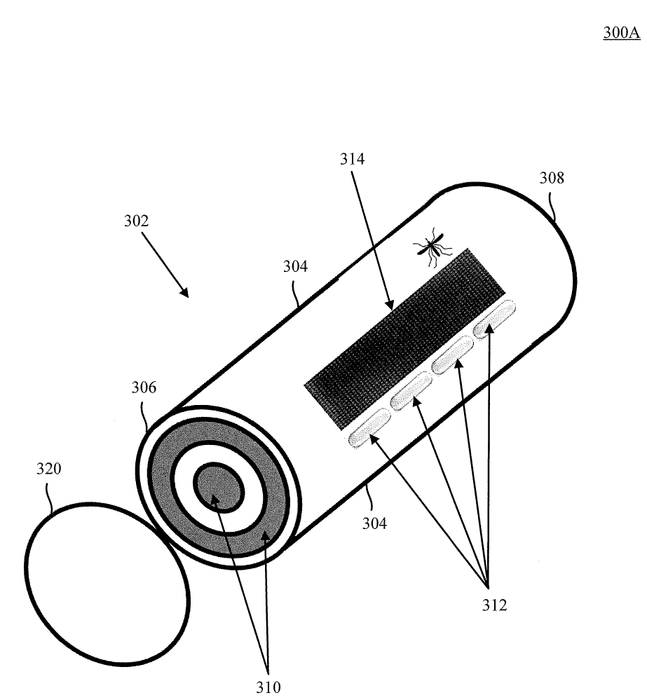

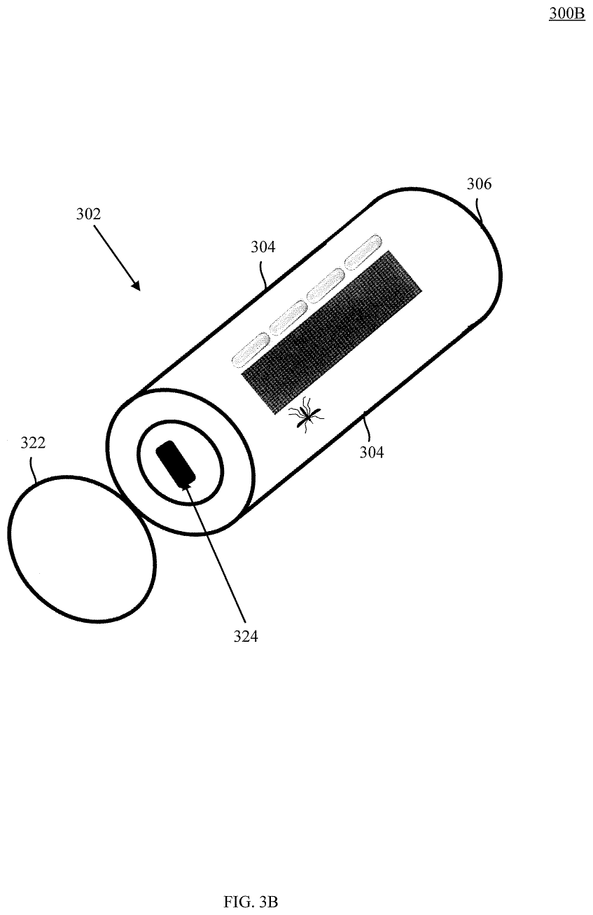

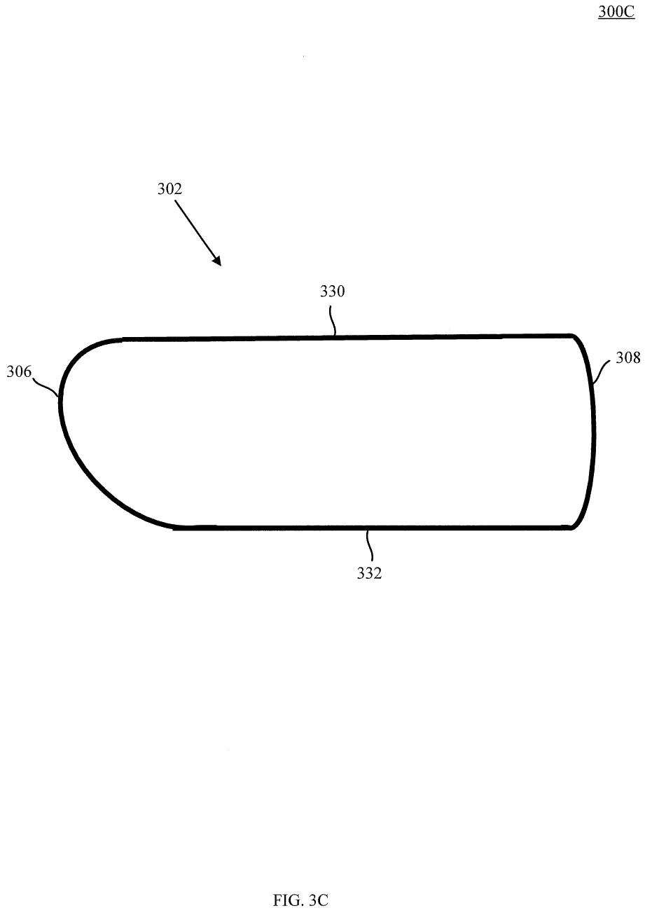

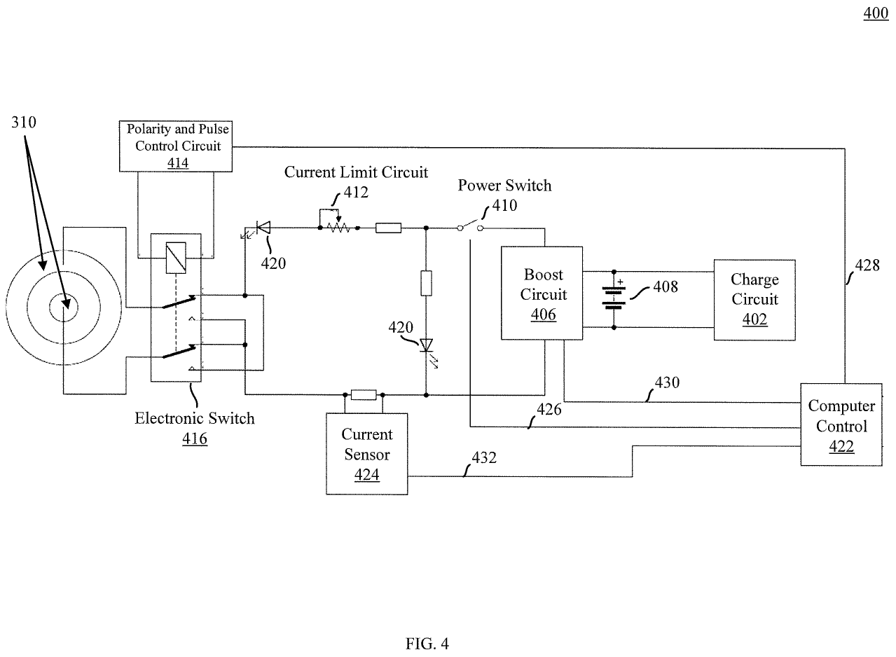

FIGS. 3A-C are diagrams 300A-C of another electrostimulation device, according to some embodiments. Like the electrostimulation device 102, an electrostimulation device 302 may include a device housing 304 that contains a circuit assembly that will be discussed in FIG. 4 and structural support. The front of the device 306 and back of the device 308 may form the exterior surfaces of the housing.

As with electrostimulation device 102, the front of device 306 may also include electrodes 310 arranged in similar configurations as electrodes 110.

Electrostimulation device 302 may include controls 312 that may be integrated with the circuit assembly and that may allow for user operation. The controls 312 may include various user interface elements such as buttons, switches, or dials for adjusting treatment parameters. In some implementations, the controls 312 may correspond to power control 112, voltage control 114, polarity control 116 and/or pulse control 118. Controls 312 may also receive instructions that select a type of an insect or snake bite. A display 314 may be positioned on the device housing 304 to provide visual feedback and information to the user in response to activating one or more of controls 312. The display 314 may show voltage settings, pulse settings, polarity settings, battery status, or other relevant information.

Like electrostimulation device 102, an electrostimulation device 302 may include a front cover 318 that can be attached to protect the electrodes 310 when the device 302 is not in use.

FIG. 3B illustrates a side and bottom view of the electrostimulation device, according to some embodiments. As illustrated in FIG. 3B, the back of device 308 may include a charge port 324. Like charge port 124, the charge port 324 may be used to connect the electrostimulation device 302 to a power source or charge an internal power source, such as a battery, or may be designed to accommodate a battery. Similarly, the electrostimulation device may include a back cover 322 that is similar to back cover 122 and is designed to cover and protect the charge port 324 when not in use.

FIG. 3C depicts a side view of the electrostimulation device, according to some embodiments. The electrostimulation device 302 may have an upper surface 330 and a lower surface 332, which may define the overall shape of the device, which may be similar to electrostimulation device 102.

FIG. 4 illustrates a circuit assembly 400 for an electrostimulation device, according to some embodiments. The circuit assembly 400 may include various interconnected components for controlling electrical stimulation delivery and enhancing the device's capabilities.

Like circuit assembly 200, the circuit assembly 400 may include a charge circuit 402 connected to a battery assembly 408. The charge circuit 402 may manage power storage and delivery for the electrostimulation device 302. A boost circuit 406 may be connected to the battery assembly 408 and may enable variable voltage control functionality. In some cases, the boost circuit 406 may include a variable voltage boost circuit, allowing for adjustable output voltages.

A power switch 410 may control the overall power delivery to the circuit assembly 400. The power switch 410 may be operatively connected to the power control on the device housing, which may turn power switch 410 on and off.

A current limit circuit 412 may be positioned to regulate the current flow through the circuit assembly 400.

A polarity and pulse control 414 may be connected to the current limit circuit 412. The polarity and pulse control 414 may manage the electrical signal delivery to the electrodes. An electronic switch 416 may be connected to the polarity and pulse control 414, controlling the routing of current between the electrodes based on signals from the polarity and pulse control 414. In some instances, polarity and pulse control 414 may be similar to polarity and pulse control 214.

Similar to light indicators 220, the circuit assembly 400 may include one or more light indicators 420 comprising an LED and associated components to provide visual feedback about the device 302 status. In some implementations, the light indicator 420 may serve as an indicator light configured to alert a user of the optimal treatment area.

A controller 422 may be connected to various components in the circuit assembly and executes instructions. For example, controller 422 may be connected to the power switch 410 via connection 428 and may activate power switch 410 in response to input via one of controls 312. Controller 422 may also be connected to the polarity and pulse control 414 via connection 428, providing computer control functionality to polarity and pulse control 414 in response to inputs via controls 312. Controller 422 may interface with multiple circuit elements, including the boost circuit 406 via connection 430, providing variable voltage control and on/off control capabilities. In some embodiments, controller 422 may receive instructions that correspond to a type of an insect or snake bite. Based on the instructions, controller 422 may access a memory (not shown) that may store various parameters, including current parameters, voltage parameters, pulse parameters, polarity parameters, and time parameters, which may be accessed and used by controller 422 to control and interface with other components in circuit assembly 400 to deliver optimal treatment based on the type of the bite. Controller 422 may also cause various commands and options to be displayed on display 312. Example commands and option may be to display applied voltage, current, time when electrostimulation device 302 was turned on, indicators to start and stop application to the bite site, and the like.

The controller 422 may also be connected to a current sensor 424 via connection 432. The current sensor 424 may detect current at electrodes 310 and provide feedback to the controller 422, allowing for adjustments to the low-voltage output based on the detected current.

The current sensor 424 may be configured to detect variations in electrical impedance across the skin surface or subcutaneous tissue. In some aspects, areas of higher impedance may correspond to the bite site or regions where venom proteins have accumulated. The current sensor 424 may utilize a low-level sensing current to measure the impedance without causing discomfort to the user or interfering with the treatment process. The current sensor 424 may also be designed to detect changes in impedance over time, which may provide information about the progression of venom spread or the effectiveness of the treatment. In some cases, the current sensor 424 may work in conjunction with the light indicator 420 to provide visual feedback to the user, guiding them to position the electrodes 310 over the areas of highest impedance for optimal treatment efficacy. In certain embodiments, the current sensor 424 may incorporate adaptive sensing algorithms that adjust the sensing parameters based on factors such as skin type, moisture levels, or depth of venom penetration. This adaptability may enhance the accuracy of bite site identification across various user demographics and environmental conditions.

The components of the circuit assembly 400 may be arranged to allow for controlled delivery of electrical stimulation to a snake or insect bity on a body part of a species, with monitoring and adjustment of current and voltage parameters to allow for continuous low voltage to be delivered via electrodes 310. As discussed above, the electrical stimulation may cause venom protein denaturation, which results in structural changes to venom proteins, resulting in loss of toxic activity. The integration of the controller 422 and current sensor 424 may enhance the capabilities of the electrostimulation device, providing adaptive treatment options based on real-time feedback from the bite site.

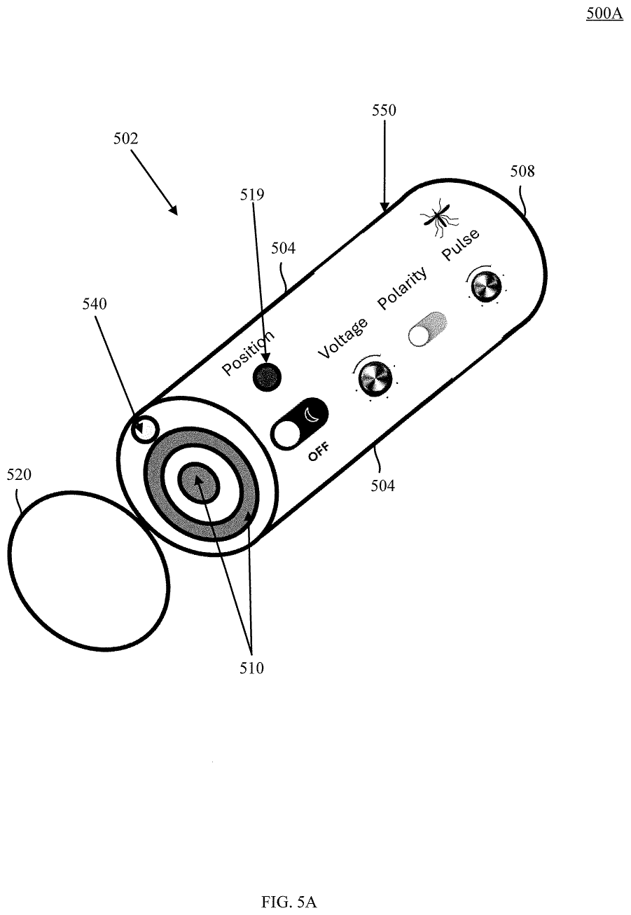

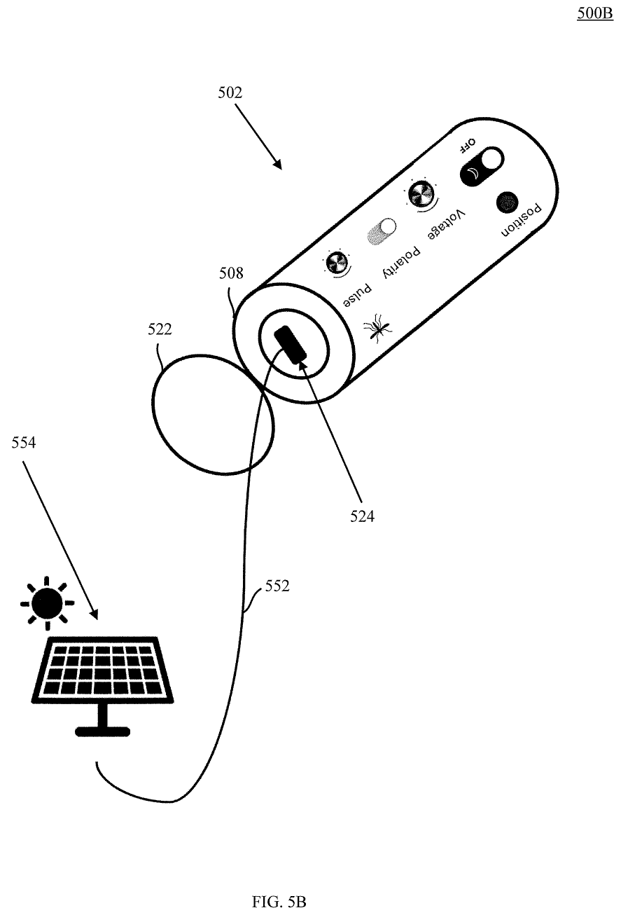

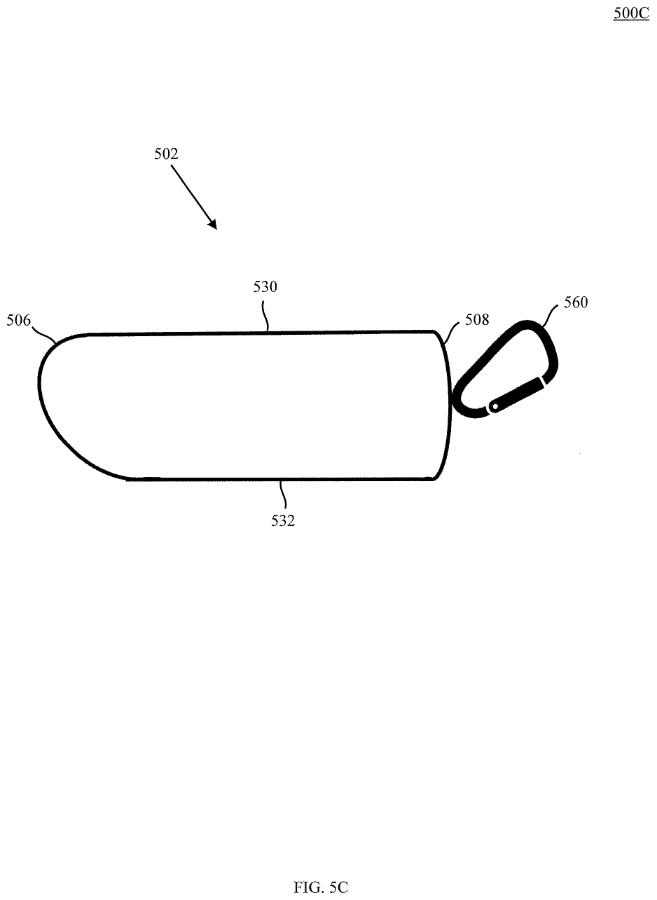

FIGS. 5A-C are diagrams 500A-C of an electrostimulation device, according to some embodiments. An electrostimulation device 502 may have a generally elongated shape with rounded edges, providing a comfortable and ergonomic form factor for handheld use, and may include features that improve usability and functionality of the electrostimulation devices. Electrostimulation device 502 may include similar components to electrostimulation device 102 discussed above.

The electrostimulation device 502 may include a waterproof electrode cover 520 designed to protect the electrodes 510 from moisture and environmental contaminants. The waterproof electrode cover 520 may be constructed from a durable, flexible material that forms a tight seal around the electrodes 510 when attached to the device housing 504. In some aspects, the waterproof electrode cover 520 may be made of a transparent or translucent material, allowing users to visually inspect the electrodes 510 without removing the cover. The waterproof electrode cover 520 may incorporate a snap-fit or threaded mechanism for secure attachment to the device housing 504, ensuring a watertight seal when in place. This design may allow the electrostimulation device 502 to maintain its water-resistant properties even in challenging environments, potentially enhancing its versatility for outdoor or aquatic use.

In some implementations, the waterproof electrode cover 520 may feature a textured surface or grip elements to facilitate easy removal and reattachment, even with wet or gloved hands. The cover may also include ventilation features that allow air circulation while maintaining water resistance, potentially reducing moisture buildup around the electrodes 510 during storage or transport.

The waterproof electrode cover 520 may be designed to accommodate various configurations of electrodes 510, ensuring compatibility with different models or versions of the electrostimulation device 502. In some cases, the cover may include conductive elements that allow for device functionality testing without removing the cover, providing an additional layer of convenience and protection for the electrodes 510.

The electrostimulation device 502 may feature an indicator light 540 that signals operational status. The indicator light 540 may use different colors or blinking patterns to communicate various device states, such as power on/off, battery level, or treatment in progress. This visual feedback may enhance user understanding and facilitate proper operation of the electrostimulation device 502. In some instances, indicator light 540 may include a UV light source described above that is located near electrodes 510. In other instances, indicator light 540 may also be an infrared heating element. The infrared heating element may be a near-infrared or mid-infrared emitter and may be applied to a bite site to deliver localized thermal energy to reduce inflammation, swelling, and/or other discomfort at the bite site. The heating element may deliver thermal energy to the bite site together with or after the low-voltage output. Indicator light 540 and the infrared heating element may be synchronized with electrical stimulation generated by electrodes 510 to simultaneously reduce microbial contamination and local inflammation at the bite site.

The electrostimulation device 502 may be similar to electrostimulation device 102, but include device housing 504 that may be made of a drop-proof material 550. The drop-proof material 550 may provide protection for the internal components particularly when electrostimulation device 502 is dropped. The drop-proof material 550 may be constructed from impact-resistant materials and may incorporate shock-absorbing features to protect the electrostimulation device 502 from damage due to accidental drops or impacts from various heights and/or a rocky or hard surface. In some cases, the drop-proof material 550 may also provide water resistance or waterproofing to enhance the device's usability in various environmental conditions.

FIG. 5B shows a side and bottom view of the electrostimulation device, according to some embodiments. The electrostimulation device 502 may show the back of device 508 which includes a back cover 522 similar to cover 122. The back cover 522 may cover a charge port 524, which may be similar to charge port 124. Additionally, charge port 524 may also be connected to a solar panel 554 via a connection cable 552. The solar panel 554 may be depicted as a flat panel structure with a grid pattern, positioned to capture solar energy for charging the device. The solar panel 554 may serve as an alternative power source, allowing the electrostimulation device 502 to be recharged in outdoor or remote settings where traditional power sources may not be available. In some cases, the solar panel 554 may be used to recharge an embedded battery, such as, but not limited to, a lithium battery within the electrostimulation device 502, enhancing the device's portability and self-sufficiency.

FIG. 5C depicts a side view of the electrostimulation device, according to some embodiments. The electrostimulation device 502 may include a front of device 506 and a back of device 508, with an upper surface 530 and a lower surface 532 defining the main body of the device. A clip 560 may be attached to the back of device 508, providing a means for securing or carrying the electrostimulation device 502. The clip 560 may allow users to attach the device to clothing, backpacks, or other gear, enhancing portability and accessibility in field situations. In some implementations, the clip 560 may be a carabiner clip, providing a secure and versatile attachment mechanism that allows users to quickly connect and disconnect the device from various objects or equipment.

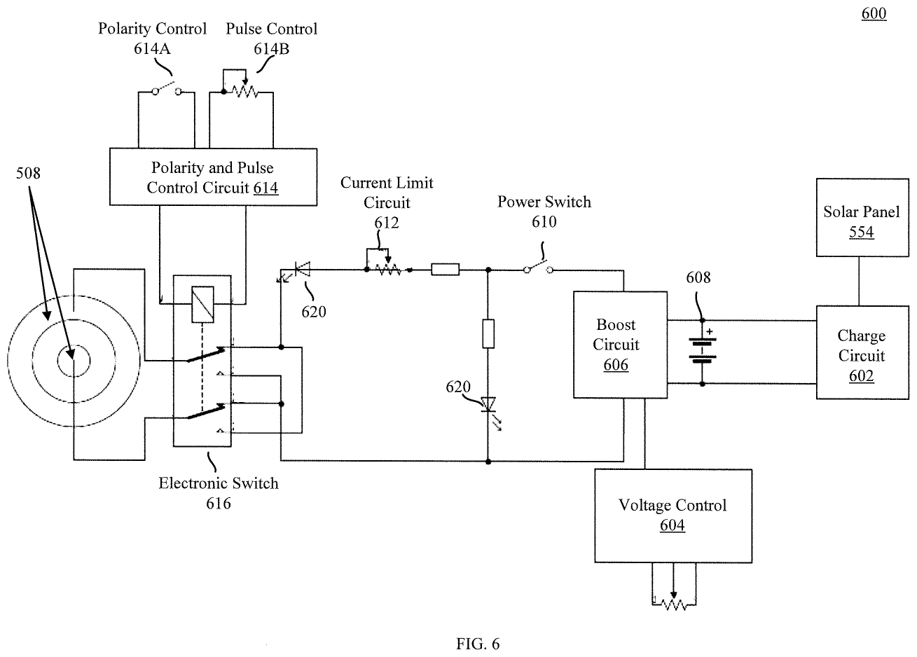

FIG. 6 illustrates a circuit diagram 600 showing an electrical configuration for a electrostimulation device with solar charging capabilities. The circuit diagram 600 depicts a charge circuit 602, a voltage control circuit 604, a boot circuit 606, a power switch 610, a polarity and pulse control 614 (including polarity control 614A and pulse control 614B), an electronic switch 616, and an LED indicator 620, which may be similar to the components discussed in FIG. 2.

Additionally, the charge circuit 602 may be connected to solar panel 554 via charge port 524. As discussed above, the connection between the charge circuit 602 and the solar panel 554 may provide an alternative power source for the electrostimulation device 502. In some aspects, this connection may allow for direct charging of the device's battery assembly using solar energy, potentially enhancing the device's usability in remote or outdoor environments where access to conventional power sources may be limited.

The charge circuit 602 may include power management components designed to regulate the input from the solar panel 554. These components may help optimize the charging process by adjusting for variations in solar intensity and converting the solar panel's output to a suitable voltage and current for the battery assembly. In some implementations, the charge circuit 602 may incorporate maximum power point tracking (MPPT) technology to extract the maximum available power from the solar panel 554 under varying environmental conditions.

The connection between the charge circuit 602 and solar panel 554 may also include protective elements such as reverse polarity protection, overvoltage protection, and surge suppression. These features may help safeguard the device's internal components from potential damage due to environmental factors or unexpected power fluctuations from the solar panel.

In some cases, the charge circuit 602 may be designed to prioritize solar charging when sufficient sunlight is available, potentially switching to battery power or alternative power sources when solar energy is insufficient. This intelligent power management may help extend the device's operational time and reduce reliance on traditional charging methods.

The integration of solar charging capabilities through the connection between the charge circuit 602 and solar panel 554 may enhance the overall versatility and self-sufficiency of the electrostimulation device, potentially allowing for extended use in field conditions or emergency situations where power availability may be uncertain.

In some embodiments, electrostimulation devices 102, 302, or 502 may also have other types of shapes and include or be coupled to a wearable band, a carabiner, and the like.

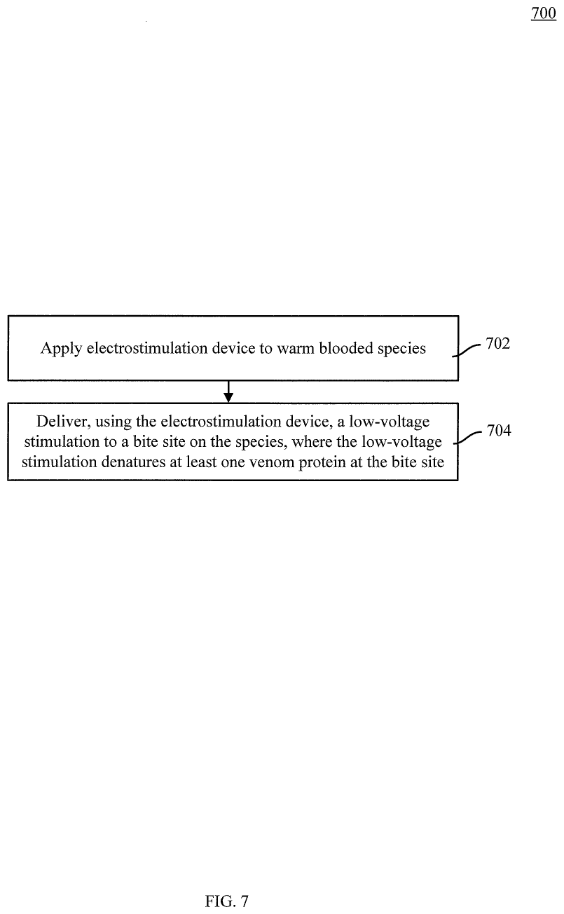

FIG. 7 illustrates a method 700 for treating venom or insect bites using an electrostimulation device, according to some embodiments. The electrostimulation device may be one of the devices discussed above with reference to FIGS. 1-6.

At operation 702, a electrostimulation device may be applied to a bite site on a warm-blooded species, such as a human, a dog, a horse, a bird, etc. In particular, the electrodes 110, 310, or 510 of respective electrostimulation devices 102, 302, or 502 may be applied to the bite site of the warm-blooded species. In some instances, one or more of electrostimulation devices 102, 302, or 502 may include a current sensor 424 and/or bite site identifier 119 that may identify a bite site or an optimal placement of the electrostimulation devices 102, 302, or 502 at the bite site by detecting current impedance at the bite site.

At operation 704, a low-voltage stimulation may be delivered through the electrostimulation to the bite site. The low-voltage stimulation may denature at least one venom protein that may be above or below a surface of the skin at the bite site. In some cases, delivering the low-voltage stimulation may comprise applying a central electrode in electrodes 110, 310, or 510 to a center of the bite site and surrounding the central electrode with a ring of electrodes. This electrode configuration may allow for targeted treatment of the affected area. In further cases, prior or during to applying delivering the low-voltage stimulation, controls 114-118 and/or controls 312 may be set to control the voltage, pulse, and polarity of the voltage that is being delivered to the bite site for providing optimum relief by breaking down the venom proteins. In some instances, the delivery of the low voltage to the bite site may be continuous as long as the electrostimulation device is applied to the bite site. In some instances, the polarity of the low-voltage stimulation may be reversed during treatment to enhance treatment effectiveness. In other cases, the low-voltage stimulation may be delivered via pulses, where each pulse may include a low-voltage output, potentially optimizing the venom protein denaturation process while minimizing tissue fatigue.

In some instances, a user treating a venomous bite in a remote setting may use the impedance sensor to identify the optimal placement zone. In this case, during treatment discussed in operation 704, the electrostimulation device may deliver low-voltage pulses to denature venom proteins, UV light to neutralize pathogens, and/or infrared heat to reduce swelling and pain at the site.

Where applicable, various embodiments provided by the disclosure may be implemented using hardware, software, or combinations of hardware and software. Also, where applicable, the various hardware components and/or software components set forth herein may be combined into composite components comprising software, hardware, and/or both without departing from the scope of the disclosure. Where applicable, the various hardware components and/or software components set forth herein may be separated into sub-components comprising software, hardware, or both without departing from the scope of the disclosure. In addition, where applicable, it is contemplated that software components may be implemented as hardware components and vice-versa.

Software, in accordance with the disclosure, such as program code and/or data, may be stored on one or more computer readable mediums. It is also contemplated that software identified herein may be implemented using one or more general purpose or specific purpose computers and/or computer systems, networked and/or otherwise. Where applicable, the ordering of various steps described herein may be changed, combined into composite steps, and/or separated into sub-steps to provide features described herein.

The foregoing disclosure is not intended to limit the disclosure to the precise forms or particular fields of use disclosed. As such, it is contemplated that various alternate embodiments and/or modifications to the disclosure, whether explicitly described or implied herein, are possible in light of the disclosure. Having thus described embodiments of the disclosure, persons of ordinary skill in the art will recognize that changes may be made in form and detail without departing from the scope of the disclosure. Thus, the disclosure is limited only by the claims.

1. A portable venom treatment device comprising:
a handheld housing having a front surface and a rear surface;
a plurality of electrodes disposed on the front surface;
a power source contained within the housing;
a voltage control circuit electrically connected to the power source and configured to generate a continuous low-voltage output; and
an electronic switch operatively connected to the plurality of electrodes and configured to selectively activate one or more electrodes to deliver the low-voltage output to a bite site, wherein the low-voltage output denatures venom proteins at the bite site.

2. The portable venom treatment device of claim 1, wherein the low-voltage output is between approximately 9V and 15V.

3. The portable venom treatment device of claim 1, wherein the plurality of electrodes comprises a concentric pattern, including a central electrode and at least one surrounding ring electrode.

4. The portable venom treatment device of claim 1, wherein the plurality of electrodes are bars parallel to each other, bars linear to each other, or multi-pin clusters.

5. The portable venom treatment device of claim 1, further comprising a polarity control circuit configured to reverse polarity of the low-voltage output.

6. The portable venom treatment device of claim 1, further comprising a pulse control circuit configured to generate a pulsed low-voltage output, wherein the pulsed low-voltage output is generated continuously for a time period.

7. The portable venom treatment device of claim 1, wherein the voltage control circuit is further configured to selectively activate the one or more electrodes in a time-dependent or impedance-responsive pattern to deliver the low-voltage output to the bite site to provide stimulation to the bite site, wherein the stimulation denatures venom proteins.

8. The portable venom treatment device of claim 1, further comprising:
a current sensor configured to detect a current impedance at the bite site; and
an indicator light configured to activate in response to the current sensor detecting the current impedance and indicate an optimal treatment area at the bite site based on the detected current impedance.

9. The portable venom treatment device of claim 1, further comprising:
an ultra-violet (UV) light source positioned adjacent to the electrodes and configured to emit ultraviolet radiation onto the bite site during generation of the low-voltage output.

10. The portable venom treatment device of claim 1, further comprising:
an infrared heating element positioned to deliver localized thermal energy to the bite site during and/or after the low-voltage output is applied.

11. A method of treating a venomous bite, comprising:
applying an electrostimulation device to a bite site on a warm-blooded species; and
delivering a low-voltage stimulation through the electrostimulation device to the bite site, wherein the low-voltage stimulation denatures at least one venom protein.

12. The method of claim 11, wherein delivering the low-voltage stimulation comprises selectively activating one or more electrodes at the bite site based on voltage signals from a voltage control circuit.

13. The method of claim 11, wherein delivering the low-voltage stimulation comprises applying a central electrode to a center of the bite site and surrounding the central electrode with a ring of electrodes.

14. The method of claim 11, further comprising:
reversing a direction of current flow between electrodes at configurable treatment intervals, wherein reversing the direction causes a change in polarity of the low-voltage stimulation.

15. The method of claim 11, further comprising:
delivering the low-voltage stimulation in pulses.

16. The method of claim 11, further comprising:
detecting current impedance at a location of the bite site; and
adjusting the low-voltage stimulation based on the detected current impedance.

17. The method of claim 11, further comprising:
detecting current impedance at a location of the bite site that is higher than other locations at the bite site or that is above an impedance threshold, wherein the location indicates the application of the electrostimulation device to a treatment area;
activating a light emitting component indicating the location as a treatment area at the bite site; and
wherein delivering the low-voltage simulation further comprising delivering the low-voltage stimulation at the location.

18. The method of claim 11, further comprising:
illuminating the bite site with an ultraviolet light during application of the low-voltage stimulation; and/or
applying infrared heat to the bite site during or after the low-voltage stimulation.

19. The method of claim 11, wherein the electrostimulation device is selected from the group consisting of a hand-held device, a solar-powered device, or a wearable band device.

20. A circuit assembly for an electrostimulation device, comprising:
a battery assembly configured to generate an input voltage;
a voltage control circuit coupled to the battery assembly and configured to set an output voltage for a low-voltage stimulation;
a boost circuit coupled to the battery assembly and the voltage control circuit and configured to modify the input voltage to generate the output voltage;
a current limit circuit connected to the boost circuit and configured to generate a current for generating the output voltage;
a plurality of electrodes comprising a central electrode surrounded by a ring of electrodes; and
an electronic switch coupled to the current limit circuit and configured to control current delivery to the plurality of electrodes, wherein the plurality of electrodes are configured to deliver low-voltage stimulation to a bite site corresponding to the output voltage, and wherein the electronic switch selectively activates the central electrode and a subset of the plurality of electrodes in a time-dependent or impedance-responsive pattern to deliver the low-voltage stimulation across the bite site.
